(19) United States
(12) Patent Application Publication
(10) Pub. No.: US 2010/0004517 A1
(43) Pub. Date: Jan. 7, 2010

(54) NON-INVASIVE METHOD AND APPARATUS FOR DETERMINING A PHYSIOLOGICAL PARAMETER

(75) Inventors: Alan Bryenton, Ottawa (CA); Izmail Batkin, Ottawa (CA)

Correspondence Address: MARKS & CLERK, P.O. BOX 957, STATION B, OTTAWA, ON, K1P 5S7, CA

(73) Assignee: BIOPEAK CORPORATION, Ottawa (CA)

(21) Appl. No.: 12/558,777

(22) Filed: Sep. 14, 2009

Related U.S. Application Data
(63) Continuation of application No. 11/055,078, filed on Feb. 11, 2005.
(60) Provisional application No. 60/543,689, filed on Feb. 12, 2004.

(51) Int. Cl. A61B 5/00

(52) U.S. Cl. 600/301

(57) ABSTRACT

The present invention relates to an apparatus and method for the non-invasive analysis of physiological attributes, such as heart rate, blood pressure, cardiac output, respiratory response, body composition, and blood chemistry analytes including glucose, lactate, hemoglobin, and oxygen saturation. Using a combination of multi-functioning disparate sensors, such as optical and electrical, improvements are made over existing physiological measurement devices and techniques. The special configuration of one or more multi-functional sensors is used to non-invasively measure multi-wavelength optical plus one or more of ECG, Bio-impedance, and RF-impedance spectroscopic data. This information is used to develop self-consistent, non-linear algorithm in order to derive the physiological attributes while compensating for various forms of interfering effects including motion artifacts, sensor attachment variability, device component variability, subject physical and physiology variability, and various interfering physiological attributes.

### CROSS REFERENCE TO RELATED APPLICATION

This application claims the benefit under 35 USC 119(e) of prior U.S. provisional application No. 60/543,689 filed Feb. 12, 2004 and is a continuation under 35 USC 120 of co-pending U.S. patent application Ser. No. 11/055,078, the contents of which are herein incorporated by reference.

### FIELD OF THE INVENTION

This invention relates to field of physiological analysis, and more particularly to apparatus and methods for the non-invasive analysis and detection of physiological characteristics, such as heart rate, blood pressure, cardiac output, respiration response and body composition including hydration, body fat content, glucose, lactate, hemoglobin and blood oxygen.

### BACKGROUND OF INVENTION

The need for the development of non-invasive physiological analysis tools stems from the prevalence in our society of obesity, lack of physical exercise, stress and demographical situation. As a result, in the US alone, more than 60 million people suffer from cardiovascular diseases, more than 18 million are diagnosed with diabetes, and more than 30% of the population is considered as overweight. Many of these people require close monitoring of physiological parameters including heart rate, blood pressure, glucose level, body index and so on.

The non-invasive analysis of physiological parameters is a very important direction of development in modern medical, consumer and fitness apparatus. Products of this type include, but are not limited to, heart rate monitors, blood pressure monitors, SpO₂ monitors, hydration and body fat monitors and so on.

From the point of view of physical principles the existing techniques can be divided in three groups: 1) the measurement of physiological parameters by using the bio-electric properties of the human body, 2) optical analysis of physiological parameters and 3) the synchronization of physiological measurements with the ECG R-peak.

The first group is based on the connection between physiological parameters and the bioelectrical properties of the human body. The most common examples of this direction include ECG detection, bio impedance monitoring of cardiac output, respiration parameters, water and fat composition, and RF glucose monitoring. Other examples of this group include EEG, EMG, EGG, nerve and muscle stimulations and so on.

One approach is based on the assumption that the glucose concentration has an effect on the complex impedance of the human body in the frequency range 1-1000 MHz, see for example, U.S. Pat. No. 5,792,668. This technique, referred to as RF spectroscopy, has been studied experimentally and applied to the design of apparatus for continuous glucose measurements inside a wristwatch. This approach has several technological advantages including low current drain and reasonably inexpensive components. The main problem with RF spectroscopy alone is that the complex impedance is sensitive to a number of factors such as water, salt, fat, temperature and so on. It is impossible to measure all those factors in real time using RF spectroscopy in order to calibrate the measurements. Therefore the use of very complicated and time-consuming calibration procedures is required. These often involve getting several invasive measurements at different glucose concentrations for comparison with RF readings so as to recalibrate the system on a regular (e.g. daily basis). Without proper regular calibration, there is no way to obtain accurate results using only RF spectroscopy.

U.S. Pat. Nos. 6,125,297 and 5,788,643, teach the use of body impedance measurements to find water and fat concentration in the human body but the results of such measurements depend on unknown salt concentration. Bio impedance measurements can provide estimates of average water and fat composition in human body but in some cases the knowledge of local body composition becomes important.

The main problem associated with bioelectrical investigation of the body's physiological parameters is the effect of other variables on the complex impedance of the human body that cannot be detected with bio-impedance measurements alone. For example, the electrolyte concentration, blood volume and so on can dramatically change the complex impedance for the same water and fat concentration.

It is known to perform optical measurements for detection of body physiological parameters. For example, U.S. Pat. No. 6,466,807 to Dobson et al teaches how to measure in vivo the concentration of an analyte using a plurality of wavelengths. U.S. Pat. No. 5,553,613 discloses a method of measuring the glucose in blood using several wavelengths. It is also known that the absorption spectrum is sensitive to the body chemistry. For example: 660 nm is sensitive to hemoglobin, 905 nm—oxy-hemoglobin, 920—fat, 970 nm—water, 1054 nm—glucose, 1253 nm—collagen, 1270 nm—water, and 1660 nm—lactate. Typically, the spectra are very broad and peaks can be shifted for different body and chemistry compositions. The actual absorption spectrum observed is the superposition of several broad bands corresponding to the individual components. It is very difficult to measure the optical path in a strongly diffuse medium such as a human body, and to extract therefrom an absolute or relative concentration of chemical components from relative measurements. It is common to use the ratios 970/810 and 1050/810 in order to find relative water and glucose concentration. The line 1050 nm contains a large contribution of water component, and the line 970 also contains contribution from collagen and fat. Therefore, there is a need to use additional information in order to separate overlapping optical bands. It is also known to synchronize optical measurements with an ECG R-peak marker.

The main problem with optical measurement and analyses is a lack of the complementary information on body parameters obtained from independent measurements.

Kiani, U.S. Pat. No. 6,526,300, teaches to combine bio-electrical measurements with optical measurements in order to ensure that a device is properly positioned and reduce the number of false alarms. In this arrangement, the electrodes are used to ensure the proper positioning of the optical sensors. They are not used in combination to measure physiological parameters.

U.S. Pat. No. 6,192,262 discloses a system for making functional maps of the human body by monitoring various physical parameters. This patent teaches that a reference parameter can be used for a choice of another parameter's recording regime, but it does not teach to improve the accuracy of a non-invasive measurement.

Additional prior art techniques involve obtaining a final result from more than one source and trying to predict the most accurate measurement, or taking a measurement and trying to compensate for changes in some perturbing factor, such as temperature, but in all such cases the final result is still in effect obtained from only one primary source of data. WO 03/063699 is an example of such a prior art technique.

### SUMMARY OF THE INVENTION

The invention takes advantage of the fact that improved results can be obtained by deriving a physiological parameter from the aggregate effect of changes in that parameter on multiple disparate physical properties. Disparate in this context means that the properties are physically different in nature. They should each be independently capable of measuring the physiological property. In accordance with the teachings of the invention, a final result is predicted from the aggregate effect of changes in the property. For example, changes in hydration level simultaneously affect optical and bio-impedance properties of an animal subject. A particular hydration level implies a particular combination of the values for optical and bio-impedance properties. By deriving the hydration level from the aggregate effect on a these properties, a more accurate result can be obtained than can be obtained from either of these properties alone or by merely attempting to compensate for inaccuracies introduced into the system, for example, by environmental changes. It will be understood in this application that the term animal refers to both human and non-human animals.

In order to obtain a measurement, calibration data reflecting the effect of changes in the physiological parameter on the physical properties need to be obtained. This can be achieved by experimentally taking measurements and creating a table and then consulting the table to obtain a parameter from a particular combination of results, or alternatively predicting the effects of changes in the physiological parameter on the properties using a mathematical model of animal physiology.

In other words, independent sources of information on body parameters should be used at the same time in order to obtain the complementary information on unknown parameters. In one embodiment optical measurements are taken as an independent source of information.

Accordingly one aspect of the invention provides a method of non-invasively determining a physiological parameter of a subject comprising generating signals representing at least two disparate physical properties of the subject, each of said disparate physical properties having a value that varies in dependence on said physiological parameter and is independently capable of giving a measurement thereof; determining the effect of changes in said physiological parameter on each of said at least two disparate physical properties; and processing said signals to derive said physiological parameter from the aggregate effect of said physiological parameter on said at least two disparate physical properties.

It will be understood in this context that the signals can be generated in any manner that creates electrical signals representing the property that are suitable for further processing. They can, for example, be generated by transducer that actively generates signals from some physical phenomenon, such as pulse rate. Alternatively, the signals could also originate within the body and be, for example, ECG signals, which are merely detected by a passive pick-up.

More than one component may be extracted from the signals during processing. For example, in the case of a complex bio-impedance the final result may depend on such values as average impedance, average phase, and average maximum rate of change of impedance.

In another aspect the invention provides a non-invasive apparatus for determining a physiological parameter of a patient comprising at least two sensors for generating and/or detecting signals representing disparate physical properties of the subject, each of said disparate physical properties having a value that varies in dependence on said physiological parameter and is independently capable of giving a measurement thereof; and a processor configured to process said signals to derive said physiological parameter from the aggregate effect of said physiological parameter on said at least two disparate physical properties.

The processor can derive said physiological parameter from calibration data stored in a memory or from a mathematical model of the animal (human or non-human) physiology.

In a preferred embodiment the at least one of the signals is optical and at least one of the other signals is an RF or bio-impedance signal. Typical physiological parameters that can be measured include water, electrolyte, fat, glucose, hemoglobin, lactic acid, cardiac output, respiration, oxygen saturation and blood pressure.

In yet another aspect the invention provides a non-invasive physiology analysis system comprising a sensor adapted for attachment to a patient and supplying to the patient an optical signal and at least one additional signal selected from the group consisting of RF and bio-impedance signals, and receiving signals from the body in response to the supplied signals; a detector coupled to said sensor for detecting said received signals and producing output signals in response to said detected signals, and a signal processing subsystem coupled to said detector and receiving said output signals, said signal processing subsystem analyzing said output signals to determine information about at least one physiology parameter.

The physiology parameter may be selected from the group consisting of water, electrolyte, fat, glucose, hemoglobin, lactic acid, cardiac output, respiration, oxygen saturation and blood pressure, and may include body composition.

The present invention therefore provides a device and methods for performing non-invasive, accurate, measurement of physiological parameters of a living body, by combining disparate technologies, such as bioelectrical and optical analysis technologies including optical spectrum analysis and one or more of bio-impedance analysis, RF impedance analysis, temperature and ECG. Specifically, the present invention can be used to measure and analyze numerous aspects of a patient's physiology, such as cardiac output, blood pressure, body composition (e.g. local and total body water, fat and electrolytes) and blood chemistry such as oxygen saturation, hemoglobin, glucose and lactate concentrations. The use of multiple inputs from disparate sources gives more accurate results than can be obtained from a single source, or a single source that is merely compensated.

### BRIEF DESCRIPTION OF THE DRAWINGS

The invention will now be described in more detail, by way of example only, with reference to the accompanying drawings, in which:

FIG. 1 is a system level block diagram of a physiology analysis system utilizing the present invention;

FIG. 2 is an equivalent circuit diagram for ECG measurements;

FIG. 3 illustrates a typical ECG signal showing R-peak;

FIG. 4 is an equivalent circuit for bio-impedance measurements of the body;

FIG. 5 is an equivalent circuit for local bio-impedance measurements;

FIG. 6 is an equivalent circuit for local RF impedance spectroscopy measurements;

FIG. 7 is a transmissive optical analysis;

FIG. 8—illustrates a backscattered/reflected optical analysis configuration;

FIG. 9 is a preferred embodiment of a two sensor module configuration;

FIG. 10 shows a minimal embodiment in the two sensor module configuration;

FIG. 11 shows a preferred embodiment in the single sensor module configuration;

FIG. 12 shows a minimal embodiment in the single sensor module configuration;

FIG. 13 shows the aggregate glucose high level process;

FIG. 14 shows the aggregate blood pressure high level process;

FIG. 15 shows the sensor attachment detection process;

FIG. 16 illustrates an ECG data acquisition process;

FIG. 17 illustrates a bio-impedance data acquisition process;

FIG. 18 illustrates an RF data acquisition process;

FIG. 19 illustrates an optical data acquisition process;

FIG. 20 illustrates a generic parameter extraction signal processing process;

FIG. 21 illustrates an aggregate glucose signal processing; and

FIG. 22 illustrates an aggregate blood pressure signal processing process in accordance with one embodiment of the invention.

### DETAILED DESCRIPTION OF PREFERRED EMBODIMENTS

As noted above, in accordance with the principles of the invention, a final result for a physiological parameter is obtained from multiple disparate sources of data.

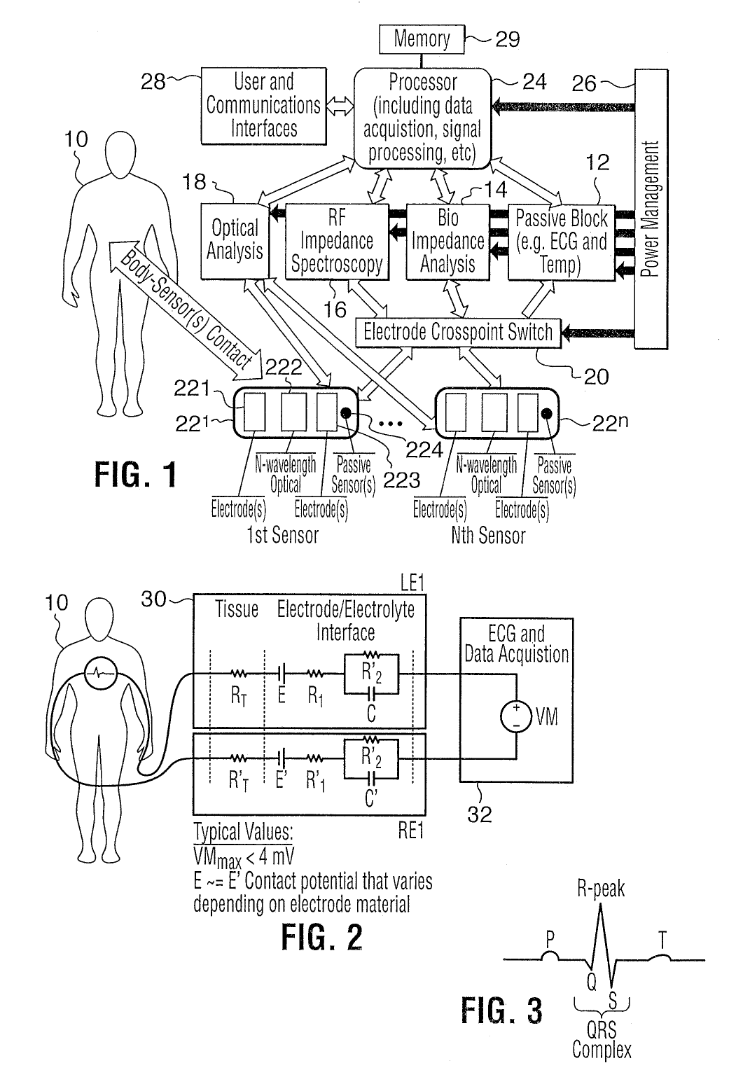

FIG. 1 discloses a system level block diagram of a preferred embodiment for analyzing the physiology of a patient 10. This system combines a physical noninvasive optical analysis subsystem with one or more physical noninvasive bioelectric measurement subsystems: a passive block subsystem that passively measures physiology attributes such as Electrocardiogram (ECG), temperature and sensor pressure; a bio-impedance analysis subsystem 14, an RF-impedance Spectroscopy subsystem; and an optical analysis subsystem 18.

An electrode cross-point switch 20 allows sensor module electrodes 22₁, . . . 22ₙ to be connected to any of the bioelectrical analysis subsystems, giving maximum flexibility in electrode configuration. The electrical cross point switch 20 allows the electrodes to be switched to a single subsystem allowing measurements to be made over an extended period or to interleave measurements from any combination of several subsystems rapidly. The cross-point switch 20 also allows multiple subsystems to be connected to the electrodes simultaneously for concurrent measurements. It would also be possible to design the system without the switch such that the electrodes are wired into one or more of the subsystems in a fixed configuration and with circuitry such as filters to allow for asynchronous and/or concurrent subsystem operations.

Outputs from the bioelectric and optical analysis subsystems are provided to the processor subsystem 24, which includes the data acquisition and signal processing functions. The data acquisition function takes analog and digital signals from the optical and bioelectrical analysis subsystems and convert them into their internal representations for further analysis. The physical implementation for the acquisition function could use any number of analog to digital converters (ADCs), digital bit-ports or integrated acquisition peripherals. However, the preferred embodiment uses an embedded processor with multiple integrated 10 and 12 bit ADCs since they are readily available and reduce the overall cost of the device. The sampling rate for the acquisition function is selected to provide sufficient resolution of the measured signals. The sampling rate and duty cycle could be different for the different sensor types.

The processor sub-system 24 may include a memory 29 storing a look-up table containing calibration data representing different values of the signals for different values of the physiological parameter in question.

The processor subsystem 24 also includes a signal processing function, which analyzes the data acquired from the optical, RF and bio-impedance analysis subsystems and the passive subsystem. The signal processing performs digital signal conditioning and statistical analysis functions such as PLS, PCR, etc. with the net result of turning the captured data into meaningful physiology attributes and other processed intermediate results. The preferred embodiment shows the data acquisition, signal processing and processor functions physically contained within the same physiology analysis device. Many combinations of components and subsystem configuration are possible depending on the technology utilized. Alternatively, they could also be physically separated in a variety of remote configurations: for example the sensor modules could be remotely connected through fiber optics and cables, the data acquisition system could transmit the raw captured data through wired or wireless communications, the signal processing function could transmit the intermediate or final results through wired or wireless communications, and the user interface could be remotely operated through wired or wireless communications. For example the raw acquired data could be sent to an external system such as a PC through a wired, fiber or BLUETOOTH™ wireless connection for analysis and/or presentation. Thus the external PC would be part of the physiology system in such a configuration.

In the preferred embodiment the processor controls the overall system and all of the subsystems either directly or indirectly. The power management subsystem 26 provides power and power conditioning for the entire system.

The user interface 28 provides interaction with the user. Input is accepted to determine what function to execute and to configure the system such as user information and calibration parameters. The user interface for a portable device could range from simple switches and LEDs to more elaborate touch screen LCD displays and keypads. The user interface for a remote system can be much more extensive such as a standalone PC based application running on a local or remote workstation, or a PDA or cellular telephone.

The device can also be accessed remotely, for example, through a network or via an attached PC through the Communications Interface, so that configuration, control, data collection, analysis and presentation can be done from a separate system and/or a separate location. USB, serial, IRDA, wireless are just a few examples of Communications Interfaces that could be used for remote access.

The sensor modules 22¹ . . . 22ⁿ are attached to the body or the body comes in contact with sensor modules so that physiology information of the body can be sensed. Each sensor 22ⁿ includes electrodes 221, multiple wavelength optical sensors 222, electrodes 223, and passive sensors 224. The physiology sensing system requires at least one sensor module containing a combination of electrodes and optical receiver/detector components. Optionally additional sensor modules may be present, each sensor module containing electrodes and/or optical components. These sensors are placed in locations sensitive to the additional information to be detected. For example, by placing an additional sensor with a pair of electrodes on the opposite side of cardiac divide from the first electrical/optical sensor, ECG, cardiac output and respiratory function information can be detected. The sensors can be conveniently mounted on a single module configured to allow the user to place a hand on the module with different fingers and the thumb exposed to different sensors.

The electrode cross-point switch 20 is used to interconnect the ECG, bio-impedance and RF-impedance spectroscopy analysis blocks to specific electrodes in the sensing modules. This switching arrangement allows any combination of two or more electrodes on any of these modules to be connected to any of the bioelectric analysis subsystems so that any combination of two electrode or four electrode configurations within a single module or between two or more modules can be configured as needed. It also allows electrodes that are not being used at a specific point in time to be left disconnected from the analysis circuitry so as to reduce power consumption and eliminate unwanted interference, which would require additional compensation circuitry to remove the interference. The electrodes can be switched to a single subsystem allowing measurements to be made over an extended period (seconds or longer) or to interleave measurements from several subsystems rapidly. The electrodes can also be connected to multiple analysis circuits simultaneously so that concurrent measurement can be made if required.

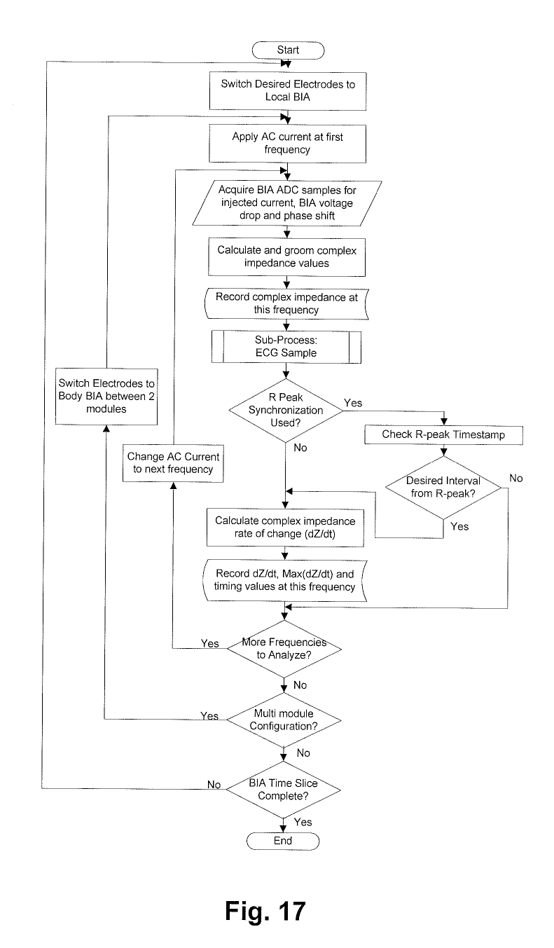

FIG. 17 illustrates how the cross point switch is used to select the correct electrodes to perform the Bio-Impedance data acquisition. The process starts by first selecting the electrodes on the primary sensor module to acquire data for local bio-impedance analysis. After the local bio-impedance analysis time slice is completed the cross point switch is used again to switch to the electrode pairs on two separate sensor modules to acquire data for body bio-impedance analysis. Note that the body bio-impedance analysis data acquisition is only performed on configurations with two or more sensor modules.

A method to automatically detect that the sensors are properly attached improves the user experience for this type of device and ensures that consistent, accurate measurements are made. The determination for proper attachment can be made from a combination of sensors in the device: the bio-impedance analysis or RF sensors for electrode connectivity, contact pressure sensor, temperature sensor and optical sensor for motion detection. For this function the bio-impedance analysis and RF sensors are used to pass an alternating current through the different electrode pairs to monitor connectivity. When the electrodes are properly attached the current will increase dramatically (to a maximum safe level) making it an ideal trigger for attachment detection. The preferred embodiment uses the bio-impedance sensors and the temperature sensor to determine proper attachment. A visual indication can be given to the user if the sensors are not properly attached, for example with a text message to the user indicating that the sensors must be readjusted. With the sensor modules properly in place, the other acquisition and analysis block functions can then start. With proper mechanical design of the outer electrodes with respect to all other sensors in the module, once the outer electrodes are determining to be properly attached, all other sensors in the module will also be properly attached.

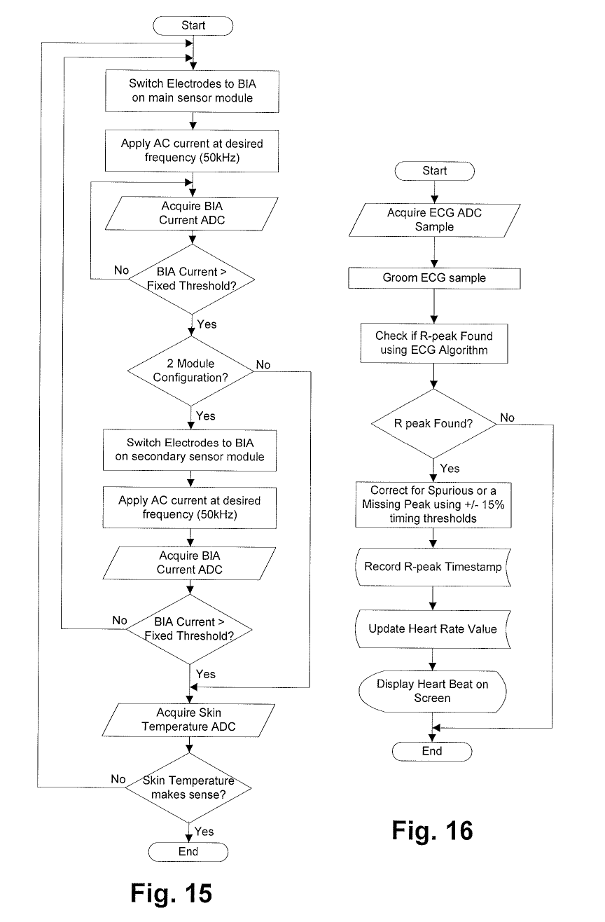

FIG. 15 illustrates the steps taken on the preferred embodiment to detect good sensor attachment before the data acquisition phases start. The same process can be used using the RF sensors for configurations without bio-impedance sensors. First the process selects the bio-impedance electrodes on the main module and applies an AC current. The AC current is monitored continuously to detect a sudden rise in current, which is expected when the sensor comes in contact with the skin. For configurations with two or more modules, this process is repeated for each sensor module. Once good contact has been detected for all sensor modules then the skin temperature can be checked to further confirm that good sensor contact has been achieved. If any of the sensor attachment checks fail then the entire process is restarted thus ensuring that all sensors are well attached at the same time.

In the passive block 12, various passive sensors can be added to help provide additional information about the target measurement site that can be used by any signal processing algorithms. For example, a thermal sensor can measure skin temperature so as to compensate for any changes that temperature might have on the other sensor readings. These passive sensors can also provide useful data directly related to the parameter of interest. Although not shown, other passive sensors such as pressure sensors to account for sensor contact pressure, humidity sensors to account for skin perspiration and/or environmental humidity, etc. could also be beneficially added. Further, passive information received from electrode pairs in separate modules can be used to pick up ECG signals.

An example of an ECG equivalent circuit 30 is shown in FIG. 2. The ECG subsystem 32 is used to pick up passive cardiac voltage potentials between an electrode on the left sensor module and an electrode on the right sensor module, for example LE1 and RE1 as shown. The raw cardiac signal is processed to determine the occurrence of R-peak as shown in FIG. 3. Most of the QRS complex spectrum is in the 5-30 Hz range and the ECG signal is very small, typically 4 mv or less. The primary function of the circuit is to isolate the QRS complex, filter out noise, especially 50/60 Hz noise and amplify the ECG signal to a range that can be properly captured by an analog-to-digital converter (ADC) in the data acquisition subsystem.

The signal is typically sampled at a rate of approximately 100 samples per second. The data acquisition sub-system extracts the following data from the ECG subsystem:
R-peak using a peak detection algorithm, as described for example in G. M. Friesen, T. C. Jannett, M. A. Jadallah, S. L. Yates, S. R. Quint, and H. R. Nagle, “A comparison of the noise sensitivity of nine QRS detection algorithms”, IEEE Trans. Biomed. Eng., vol. 37, pp. 85-98, January 1990.
Statistic on timing and interval of R-peaks are analyzed so that false R-peak detects and missed R-peaks are adjusted for.
Heart rate calculated from the time between R-peaks. The heart rate is typically averaged over a 5 second moving window to act as a damper to heart variability and to filter out possible invalid and missed R-peak detections.

FIG. 16 illustrates how ECG samples are acquired and processed. The ECG data acquisition process is designed to operate concurrently with the bio-impedance, RF and optical data acquisition processes so that these processes can be run independently or synchronized with the ECG R-peak. The electrodes on the preferred embodiment are permanently connected to the ECG subsystem therefore it is not necessary for the cross point switch to connect the electrodes to the ECG. Configurations without permanent ECG connections will require the electrodes to be connected to the ECG subsystem. A single ECG sample is acquired and groomed using a digital filter to be used in the R-peak search algorithm. See reference [QRS] “A comparison of the noise sensitivity of nine QRS detection algorithms” for a description of nine different peak search algorithms. If an R-peak is found then a time stamp is taken for use by the bio-impedance, RF and Optical data acquisition processes for synchronization. The heart rate is also updated and displayed on screen.

Bio-Impedance is defined herein to cover the frequency range from 0 Hz to 1 MHz and RF is defined herein to cover the range from 1 MHz and higher. This distinction has been made due to the different circuitry required for these ranges and the different types of information found in each range.

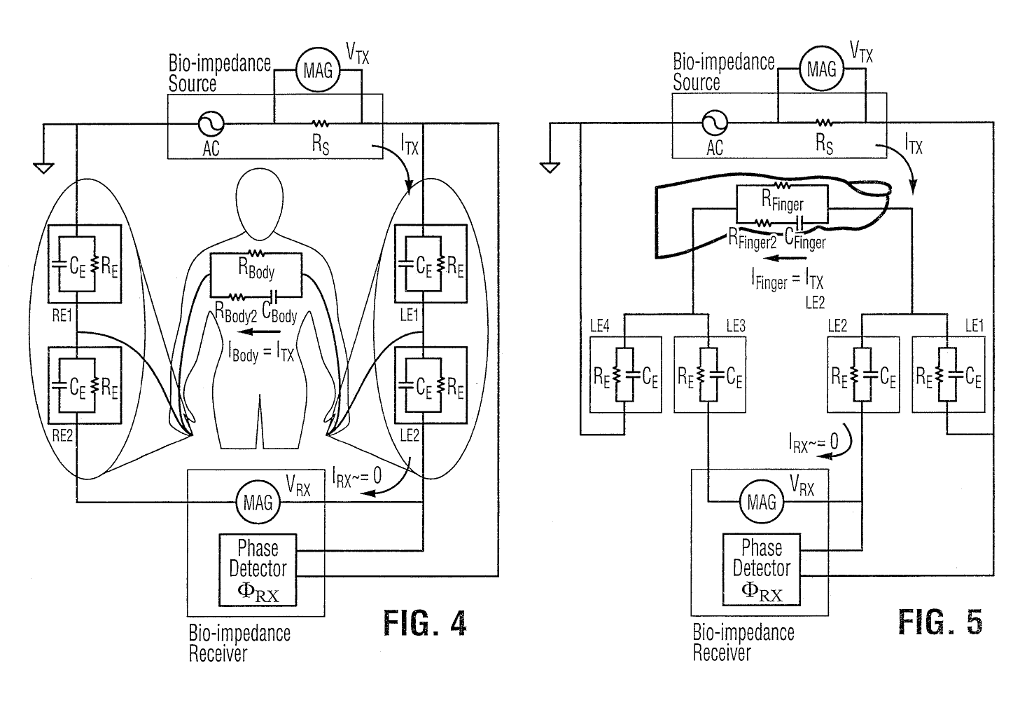

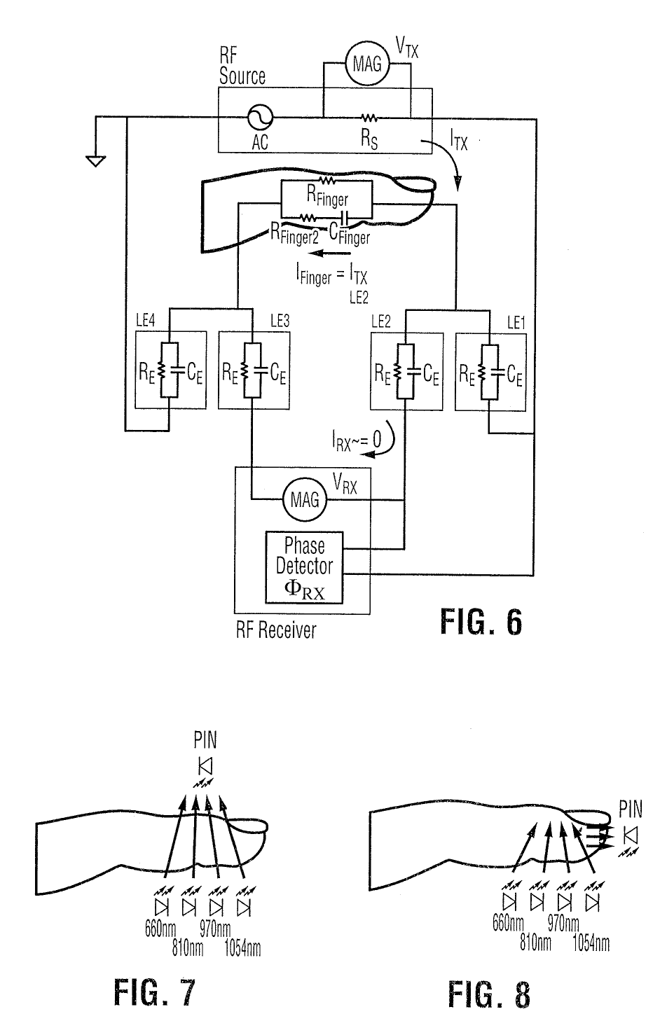

The Bio-impedance sub-system is used to inject alternating current in the sub MHz range into the body between electrodes on two separate sensor modules as shown in FIG. 4. Preferably the source supplies less than 1 mA (for safety) of sinusoidal current at several frequencies in the range of 1 Hz to 100 kHz and less than 10 mA in the range of 100 kHz to 1 MHz. The bio-impedance subsystem measures the complex impedance across the body (between electrodes in separate sensor modules—as shown in FIG. 4) or across the local body part (between electrodes within a single sensor module—as shown in FIG. 5). Different current levels and periodic waveforms can be used to perform a similar bio-impedance function. The resultant phase and magnitude information from the Bio-impedance block is sampled by the data acquisition system so that it can be used by the signal processing function to calculate body composition information such as local and body water content, local and body electrolyte content and local and body fat content etc.

The Bio-impedance circuit can be connected to electrodes simultaneously with the ECG sub-system. This allows the signal processing function to use the ECG R-Peak to synchronize the Bio-impedance measurements to improve the bio-impedance signal processing by focussing the processing to a specific interval in the cardiac period.

The bio-impedance analysis sub-system measures the complex impedance across the body or across a local tissue area. One method of determining complex impedance is using the theory of AC phasors. By injecting a sinusoidal waveform into the body the magnitude of the complex impedance can be determined and the phase angle can be determined using a phase detector.
The current being injected into body (I_{Body}) is derived by measuring the voltage (V_{Tx}) across a series source resistor (R_{S}).

$${I}_{Body} = \frac{{V}_{Tx}}{{R}_{S}}$$

The complex impedance magnitude of the body (Z_{Body}) is calculated by measuring the current flowing through the body (I_{Body}) and measuring the voltage drop across the body (V_{RX}) (i.e. ohm's law).

$$\vert {Z}_{Body} \vert = \frac{{V}_{Rx}}{{I}_{Body}}$$

The voltage drop across the body (V_{RX}) is measured through a second set of electrodes (RE2 and LE2). The electrode resistances (R_{E}) do not affect the voltage measurement since the high input impedance of the magnitude and phase detectors draws virtually no current.

The phase shift (Φ_{RX}) of the injected signal with respect to the received signal is measured using a phase detector.

The real and imaginary parts of the complex impedance can be determined using the following formula:

Z_{Body}=|Z_{Body}|<φ_{RX}=R+jX=|Z_{Body}|cos(φ_{RX})+j|Z_{Body}|sin(φ_{RX})

The body impedance is derived from the current and voltage drop across the body. A constant current source could be used for the measurement eliminating the need to measure the current. However, in this embodiment, a measured current method is used. This method requires an additional ADC to measure the voltage drop across a reference resistor to derive the injected current. Phase is extracted using a phase detector and is acquired through an ADC.

The device acquires all or part of the following data during a fixed acquisition period:
Average Impedance (Real): the average real impedance is calculated. However it may be sufficient to measure the average magnitude, which avoids having to calculate the real impedance from the raw impedance measurement.
Average Phase
Average Max (dZ/dt): This value can be synchronized with the ECG R-peak to increase the reliability of detecting dZ/dt peaks vs. other artifacts. The maximum dZ/dt typically occurs 200-400 ms through an R-peak to R-peak cycle. This dZ/dt value is averaged over the acquisition period.
Average Time from R-peak to Max (dZ/dt) if R-peak synchronization is used.

Bio-impedance can also be measured locally between electrodes in a single sensor module as shown in FIG. 5. The complex impedance information is used to derive local water, electrolyte and fat information. The voltage drop across the local tissue (V_{RX}) is measured through a second set of electrodes (LE2 and LE3). The electrode resistances (R_{E}) do not affect the voltage measurement since the high input impedance of the magnitude and phase detectors draws virtually no current.

FIG. 17 illustrates how the Bio Impedance data is acquired for use in the final parameter signal processing algorithms. The same process is used to acquire the bio-impedance data for local (single module) and body (multi module) measurements at a number of frequencies. First the bio-impedance electrode pairs are selected and an AC current is injected into the tissue. The injected signal is recovered and the tissue complex impedance is derived from the raw voltage, current and phase shift measurements (using ohm's law). Instantaneous and average complex impedance is recorded. Then the rate of change of the complex impedance (dZ/dt) is computed to find the maximum rate of change (max (dZ/dt)) and the time interval from R-peak to max (dZ/dt) (if R-peak synchronization is used). These values are recorded for use in the final data processing algorithms. If R-peak synchronization is used then the dZ/dt, max (dZ/dt) and timing measurements calculations are skipped unless the sample is taken during the desired time interval from R-peak. The acquisition process is repeated for each frequency and set of electrodes. The bio-impedance subsystem must wait for the injected signals to stabilize before making measurements, which makes it difficult to switch rapidly to and from the bio-impedance subsystem. For this reason the bio-impedance data acquisition process is given an appropriate time slice to complete all of its measurements.

The RF-impedance Spectroscopy block, as shown in 6, is used to inject RF frequency alternating current into the body between a pair of electrodes at a single site in a single sensor module. The source supplies a sinusoidal current at several frequencies in the range of 1 MHz to 5 GHz and measures the phase and magnitude across the local body part between the electrode pair. For safety, the injected current is limited to a maximum safe level. Different current and periodic waveforms could be used to perform a similar RF-impedance spectroscopy function. The resultant phase and magnitude information from the RF-impedance spectroscopy block is sampled by the data acquisition system so that signal processing can be performed to determine local composition information such as water, electrolyte and glucose content. The sampling of the RF signal can be referenced with other strong periodic signals such as R-peak or photo-plethysmograph. This time referencing is useful to increase the recovered signal quality and can also be used to more accurately measure RF-impedance at the peaks and troughs of the cardiac pulse. These peak and trough measurements can then be used to perform RF pulse spectroscopy, a novel technique of the present invention to isolate arterial blood RF spectral information.

RF pulse spectroscopy uses a technique similar to optical pulse oximetry but uses the ratio of AC to DC RF impedance at one frequency compared to the RF impedance ratio at one or more other frequencies. The benefit of this technique is that the non-arterial impedance components such as tissue, venous blood, fat, etc that are constant in both measurements can be cancelled out, and allows isolation of arterial blood component RF effects.

The RF circuit operates in parallel to the ECG circuit since it can beneficially use the ECG R-Peak to synchronize measurements. The phase and impedance are measured at multiple RF frequencies on one location only. The RF Impedance Spectroscopy hardware design differs from the Bio Impedance hardware in that it requires higher frequencies (greater than 1 MHz), and it is measured across local body part only (e.g. a finger, wrist or forearm).

The RF Impedance Analysis Subsystem acquires all or part of the following data:
Instantaneous and Average Impedance at each frequency.
Instantaneous and Average Phase shift at each frequency.
Arterial pulse peak and trough complex impedance at each frequency. This measurement can be synchronized to the ECG R-peak to enhance peak determination and accuracy.
Rate of change of impedance over time (dZ/dt) at one or more frequencies.
Maximum rate of change of impedance, Max (dZ/dt), at one or more frequencies.
Instantaneous and Average Time from R-peak to Max (dZ/dt) at one or more frequencies.

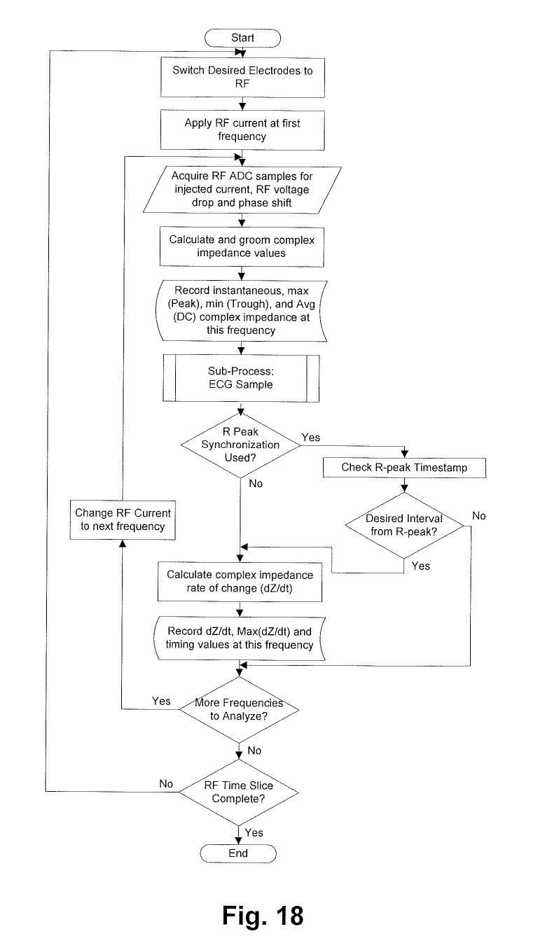

FIG. 18 illustrates how the RF data is acquired for use in the final parameter signal processing algorithms. First the RF electrode pairs are selected and an RF current is injected into the tissue. The injected signal is recovered and the tissue complex impedance is derived from the raw voltage, current and phase shift measurements (using ohm's law). Instantaneous and average complex impedance are recorded. Then the rate of change of the complex impedance (dZ/dt) is computed to find the maximum rate of change (max (dZ/dt)) and the time interval from R-peak to max (dZ/dt). These values are recorded for use in the final data processing algorithms. If R-peak synchronization is used then the dZ/dt, max (dZ/dt) and timing measurements calculations are skipped unless the sample is taken during the desired time interval from R-peak. The acquisition process is repeated for each RF frequency resulting in a discrete complex impedance spectrum. The RF subsystem must wait for the injected signals to stabilize before making measurements, which makes it difficult to switch rapidly to and from the RF subsystem. For this reason the RF data acquisition process is given a time slice to complete all of its measurements. The time slice size depends on the configuration and the number of frequencies being measured.

The Optical Analysis block 18 injects light into the body and detects absorption and scattering of the light at 1 or more optical wavelengths. The wavelengths used in the present embodiment are in the visible-NIR range from 400 nm to 2500 nm, although UV, MIR, FIR and other wavelengths that exhibit good transmission properties through the skin and have discernible absorption and/or scattering by chemicals or tissue of interest, could also be used. The optical subsystem light source is designed to handle one or more LEDs. However, laser diodes, or other light sources that produce sufficient light in the wavelength bands of interest could equally well be used. The output intensity and shape of the light source are set to maximize recovered signal for the specific frequency and configuration. The light source is positioned so as to illuminate the subject's finger or other body part in which light absorption of the blood can be detected. One or more detectors that are sensitive to light in the wavelengths required for the specific application are used to collect light in either a transmissive and/or reflective/backscattered configuration. Alternate source-detector arrangements can be used so long as sufficient power at the necessary wavelengths for the specific application can be detected. For example, incandescent or halogen light bulbs can be used with narrow band filters at the specific frequencies of interest. For wavelengths above about 1100 nm, some form of shutter or pulsing mechanism may also be required to provide for sufficient NIR energy emission during the illumination period, but block off the light for the remainder of the period to protect the skin and tissue from thermal injury.

The sampling of the optical signals can be referenced with other strong periodic signals such as R-peak or photo-plethysmograph signals. This time referencing is useful to increase the recovered signal quality and can also be used to more accurately measure optical absorption and scatter at the peaks and troughs of the cardiac pulse. These peak and trough measurements can then be used to perform optical pulse spectroscopy to isolate arterial blood optical spectral information. The resultant optical information from the Optical Analysis block is sampled by the data acquisition system so that signal processing can be performed to determine local composition information such as water, haemoglobin, oxygen saturation, blood glucose, lactate and others.

Many Visible—Infra-Red (IR) sensors today are transmissive: they shine light from one side of the finger (or earlobe, toe, etc.) and detect the light on the other side, as shown in FIG. 7. The major disadvantage of transmissive spectroscopy is that it is mechanically more difficult to design. The photo detectors need to be built into the outside mechanical structure, which means that separate electronic module and cabling are needed. Additionally, the range of tissue types and finger sizes etc. that need to be accommodated tends to make calibration difficult. The big advantage of using transmissive optics is that it is possible to do a calibration of the optics before the finger is inserted. When the LED is turned on, the received light signal is measured without anything in the light path. This effectively calibrates out any aging effects of the LEDs and photo detectors as well as dust, scratches, etc. on the lenses.

Reflective spectroscopy, as shown in FIG. 8 is easier to implement mechanically. The LED and photo detectors can both be built into the same electronic module in the main device housing. The challenge of reflective spectroscopy is that the optics are somewhat more difficult to calibrate after the device is in the field. There are also issues with isolating the photo detector from the light source since they are in such close proximity. This can be solved by using some sort of baffle or by using a lens to ensure that the light goes directly into the finger. By tapping off a portion of the emitted light energy for each of the frequencies, for example with a 1:100 prism, the transmission energy of each of the frequencies can be determined and from this the relative emission energies at each frequency. These emission energies can be used to normalize each of the recovered reflective/backscattered optical signals so that the ratios of absorption/scattering of each frequency can be determined.

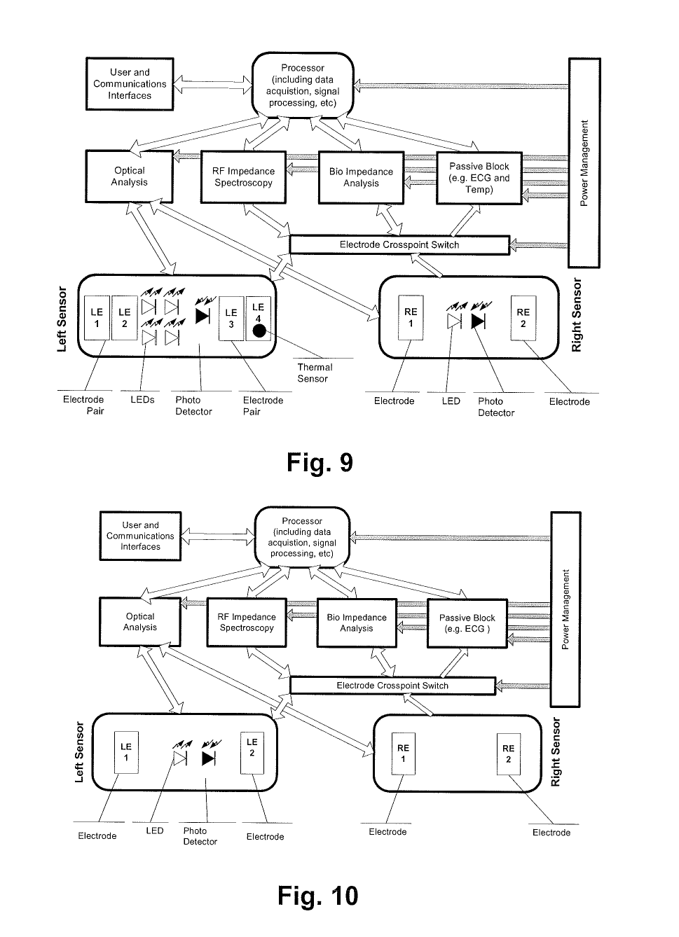

FIGS. 8 and 9 illustrate light injected at different frequencies, for example 660 nm, 810 nm, 970 nm, 1054 nm due to their sensitivity to haemoglobin absorption, haemoglobin isobestic point, water absorption and glucose absorption respectively. More or less than 4 frequencies as well as other frequencies could equally well be used without changing the intent of the current system.

The optical analysis subsystem acquires all or part of the following data:
1. Average energy at each wavelength without subject in place (Reference measurement)
2. Average energy (DC) at each wavelength with subject in place
3. Arterial pulse Peak and Trough energy (AC) at each wavelength with subject in place. Synchronization with R-Peak can optionally be used to improve the determination of these values.
4. Average Max (dI/dt) at one or more frequencies with subject in place. This can be synchronized with the ECG R-peak to improve accuracy. It involves measuring the maximum dI/dt, which typically occurs 200-300 ms after R-peak. This value is averaged over the acquisition period.
5. Average Time from R-peak to Max (dI/dt) at one frequency only with subject in place.

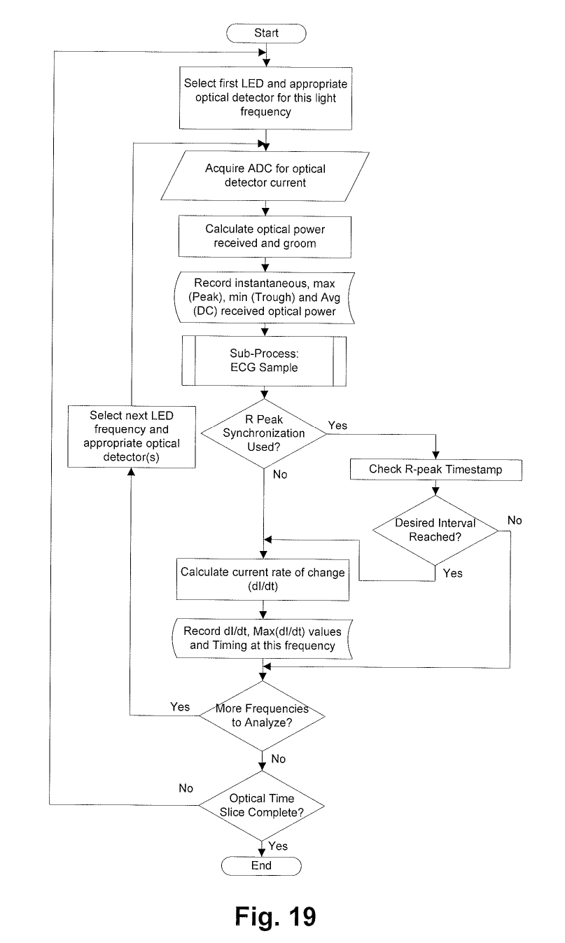

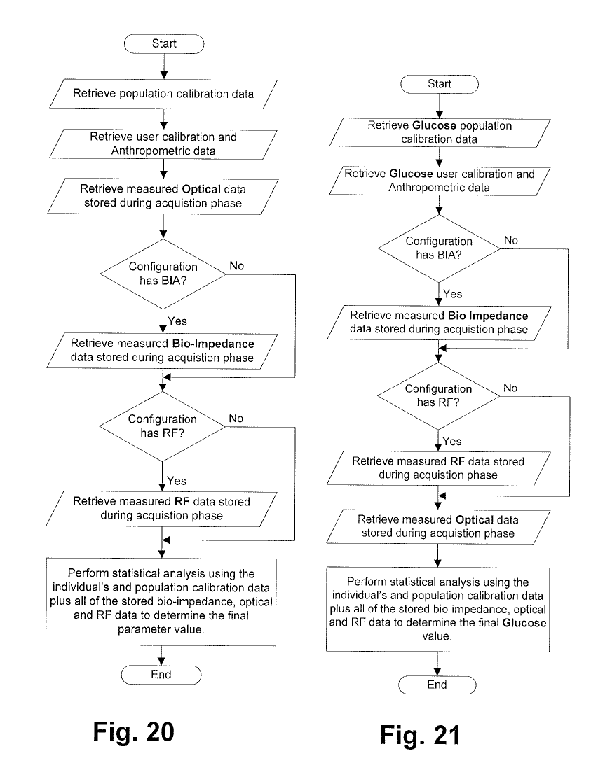

FIG. 19 illustrates how the Optical data is acquired for use in the final parameter signal processing algorithms. The first LED and the associated optical detector are selected. A short burst of light is produced and the received optical power is acquired and groomed from the raw optical detector current. Instantaneous and average optical received powers are recorded. Then the rate of change of the optical power (dI/dt) is computed to find the maximum rate of change (max(dI/dt)) and the time interval from R-peak to max(dI/dt). These values are recorded for use in the final data processing algorithms. If R-peak synchronization is used then the dI/dt, max (dI/dt) and timing measurement calculations are skipped unless the sample is taken during the desired time interval from R-peak. The acquisition process is repeated for each optical frequency. The optical data acquisition process is given a time slice to complete all of its measurements. The time slice size depends on the configuration and the number of frequencies being measured.

Since many of the sensors are measuring interdependent or identical attributes, self consistency between identical attributes can be performed to ensure that the most accurate information is determined, and corrections for interfering attributes can be made. For example, water concentration can be determined using local and body bio-impedance, optical analysis and by using RF-impedance Spectroscopy. However RF water measurements are shifted by electrolyte concentrations, which are not easy to isolate in the RF domain, and optical water measurements are impacted by lactate and other blood chemical concentration changes. Since bio-impedance can isolate electrolyte from water content (1 kHz vs. 50 kHz) to give accurate estimates of each, this information can be used by both optical and RF to correct for water and electrolyte contributions. In a similar fashion both optical and RF can detect glucose but water and electrolyte interfere in RF measurements and water and lactate interfere in Optical. So using bio-impedance, water and electrolyte corrections, both optical and RF can improve determination of glucose concentrations. These adjustments are repeated with the new refined measurements until the water, electrolyte, lactate and glucose concentration information from each subsystem is as accurate as the system will allow.

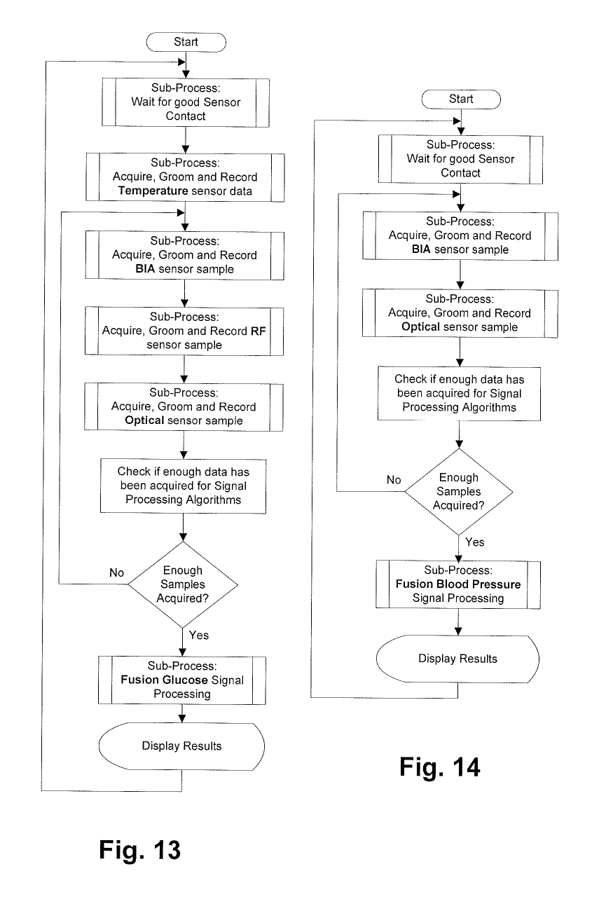

FIG. 13 shows a typical sequence of how a physiological parameter is analyzed from multi-sensor information. In this example glucose is measured in the blood non-invasively by acquiring data from Bio-impedance, RF and Optical sensors that is then processed and displayed to the user.

FIG. 21 shows how the acquired bio-impedance, RF and optical data are used in conjunction with population calibration data and user calibration data to derive the final Glucose parameter value.

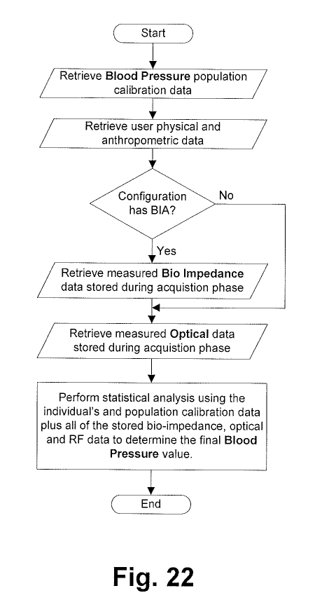

FIGS. 14 and 22 show another example for blood pressure measurements.

A wide range of physiological parameters can be derived using procedures similar to the Glucose and Blood pressure described above. The physiological parameters include, but are not limited to, lactate, body water, body fat, body electrolytes, local tissue water, local tissue fat, local tissue electrolytes, cardiac output, cholesterol, etc.

FIG. 9 shows a preferred two sensor module configuration. The modules can be located in a variety of places such as fingers, wrists or forearms, ideally, but not restricted to, where there is plenty of vascular blood in the underlying tissue as well as a detectable arterial pulse. Sensor modules must be placed on opposite sides of the cardiac divide to be able to pick up cardiac and respiratory information. The left sensor module contains 4 high conductivity electrodes, 2 or more LEDs in the visible-NIR range, detector(s) sensitive to the transmitted wavelengths and a thermal sensor. Typical wavelengths chosen are those sensitive to attributes of interest. For example, 970 nm is sensitive to water, 810 nm since it is equally sensitive to oxygenated and deoxygenated haemoglobin (i.e. haemoglobin isobestic point), 1054 nm for sensitivity to glucose, 660 nm for higher sensitivity to deoxygenated vs. oxygenated haemoglobin and 1660 nm for sensitivity to lactate. Other wavelengths, with sensitivities to other physiology attributes could also be used. The detector(s) are chosen such that they are sensitive to those wavelengths and to pick up energy at the desired locations. For example, a single Silicon detector could be used to cover wavelengths from roughly 500 nm-1100 nm, an InGaAs detector could be used to cover the range from roughly 900 nm-1900 nm or multiple detectors could be used to pick up both reflective and transmissive energies and/or cover the range from 500 nm-1900 nm. The right sensor module contains 2 high conductivity electrodes, a single LED that emits in the visible-NIR range and a detector that is sensitive to the single LED's transmitted wavelength. The LED wavelength such as 660 nm is chosen to allow detection of a strong photo-plethysmograph signal. In such a configuration all of the analysis subsystem functions can be performed, allowing blood pressure; cardiac output; respiratory function; local and body water, fat and electrolytes; and blood chemistry attributes to be determined.

FIG. 10 shows the minimal configuration for a 2 Sensor Module system. This configuration accommodates a 4-wire bio-impedance circuit to measure body composition, a 2 electrode ECG to measure cardiac output and respiratory functions and a simple optical source and detector with a single LED. The optical source and detector can be used to implement a photo-plethysmograph as well as determine tissue scattering properties and relative absorption properties at a pair of wavelengths which can be used to determine oxygen saturation or measure other blood chemistry attributes. Additionally blood pressure can be determined by analyzing the timing relationship between the ECG and the photo-plethysmograph.

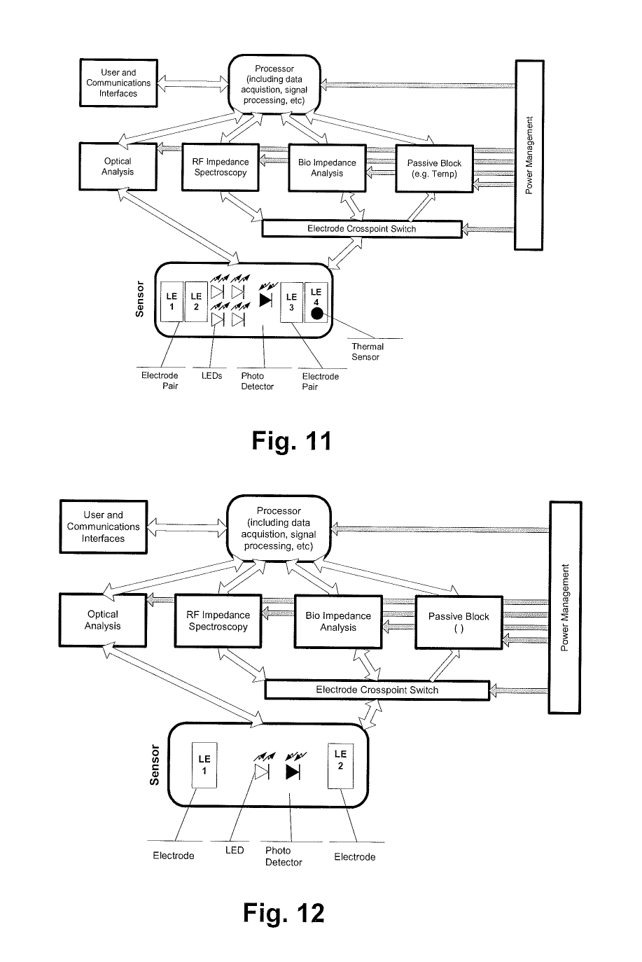

FIG. 11 shows the preferred configuration for a single sensor module system. The sensor module contains four high conductivity electrodes, two or more LEDs in the visible-NIR range, detector(s) sensitive to the transmitted wavelengths and a thermal sensor. The choice of number and wavelengths of LEDs and the number and frequency of detector(s) depends on the specific application and sensor location, as described previously. In such a configuration optical, RF and local bio-impedance analysis subsystem functions can be performed, allowing blood pressure; local water, fat and electrolytes; and blood chemistry attributes to be determined.

FIG. 12 shows the minimum configuration for a single sensor module system. The sensor module contains 2 high conductivity electrodes, 1 LEDs in the visible-NIR range and a detector sensitive to the transmitted wavelengths. The choice wavelengths of LEDs and detector depend on the specific application and sensor location, as described previously. In such a configuration optical, RF and local bio-impedance analysis subsystem functions can be performed, allowing blood pressure; local water, fat and electrolytes; and blood chemistry attributes to be determined.

The following Table summarizes the various attributes that each configuration can provide and an indication of which technique is best when there is a difference.

| | Minimum 1 | Preferred 1 | Minimum 2 | Preferred 2 |
|---|---|---|---|---|
| | Sensor | Sensor | Sensor | Sensor |
| Attribute | Module | Module | Module | Module |
| Heart rate | ✓ | ✓ | ✓-best | ✓-best |
| Cardiac | | | ✓ | ✓ |
| Output | | | | |
| Blood | ✓ | ✓ | ✓ | ✓-best^{A} |
| Pressure | | | | |
| Respiratory | | | ✓ | ✓ |
| Function | | | | |
| Local | ✓ | ✓-best | ✓ | ✓-best |
| electrolytes | | | | |
| Local water | ✓ | ✓-best | ✓ | ✓-best |
| Local fat | ✓ | ✓-best | ✓ | ✓-best |
| Body | | | ✓ | ✓-best^{B} |
| electrolytes | | | | |
| Body water | | | ✓ | ✓-best^{B} |
| Body fat | | | ✓ | ✓-best^{B} |
| Blood glucose | | ✓ | | ✓-best |
| Blood | | ✓ | | ✓-best |
| Oxygen | | | | |
| Saturation | | | | |
| Blood lactate | | ✓ | | ✓-best |
| Other Blood | | ✓ | | ✓-best |
| attributes | | | | |

In the above table superscript A indicates: ECG sync, BIO-IMPEDANCE valve open detect and single or dual PPG PTT. Superscript B indicates 4-wire local composition corrections were used.

We claim:

1. A method of noninvasively measuring a global body level physiological parameter of a subject comprising:
a) locating respective noninvasive sensors on the body of the subject, said sensors outputting separate signals representing respective measured values of at least two disparate measurable physical properties of the subject, the value of each of said disparate physical properties varying in dependence on said physiological parameter and being independently capable of giving a measurement of said body level physiological parameter;
b) obtaining calibration data reflecting the effect of changes in said physiological parameter on the values of each of said at least two disparate measurable physical properties so as to permit the identification of the values of said body level physiological parameter corresponding to particular combinations of values of said disparate measurable physical properties;
c) processing said signals representing said respective measured values of said disparate physical properties in a processor configured to transform said signals into a final value signal representing a quantitative measurement of said body level physiological parameter that is derived from a plurality of said signals and based on the particular combination of the measured values of said disparate properties represented by said signals, whereby said final value is based on the aggregate effect of changes in said physiological parameter on said at least two disparate physical properties predetermined in step b); and
d) outputting said final value of said body level physiological parameter.

2. An apparatus for noninvasively measuring a global body level physiological parameter of a subject comprising:
a) noninvasive sensors for location on the body of the subject, said sensors outputting separate signals representing respective measured values of at least two disparate measurable physical properties of the subject, the value of each of said disparate physical properties varying in dependence on said physiological parameter and being independently capable of giving a measurement of said body level physiological parameter;
b) a memory storing calibration data reflecting the effect of changes in said physiological parameter on the values of each of said at least two disparate measurable physical properties so as to permit the identification of the values of said body level physiological parameter corresponding to particular combinations of values of said disparate measurable physical properties;
c) a processor configured to processing said signals representing said respective measured values of said disparate physical properties so as to transform said signals into a final value signal representing a quantitative measurement of said body level physiological parameter that is derived from a plurality of said signals and based on the particular combination of the measured values of said disparate properties represented by said signals, whereby said final value is based on the aggregate effect of changes in said physiological parameter on said at least two disparate physical properties predetermined in step b); and
d) a display for outputting said final value of said body level physiological parameter.
